(19) United States
(12) Patent Application Publication
(10) Pub. No.: US 2007/0117221 A1
(43) Pub. Date: May 24, 2007

(54) Dielectrophoretic controlled scat hormone immunoassay apparatus and method

(76) Inventors: Alex Nugent, Santa Fe, NM (US); Timothy Molter, Saukville, WI (US); Partick McVittie, Palo Alto, CA (US); Matthew P. Horning, Seattle, WA (US)

Correspondence Address: ORTIZ & LOPEZ, PLLC;Patent Attorneys, P.O. Box 4484, Albuquerque, MN, 87196-4484, US

(21) Appl. No.: 11/453,619

(22) Filed: Jun. 15, 2006

Related U.S. Application Data
(60) Provisional application No. 60/691,699, filed on Jun. 16, 2005.

(51) Int. Cl. G01N 33/543; C12M 3/00

(52) U.S. Cl. 436/518; 436/523; 435/287.2

(57) ABSTRACT

An immunoassay apparatus on a chip is disclosed, which can quantitatively measure the concentration of hormones (particularly corticosterone and progesterone) in a biological sample. Such an apparatus can be designed to be used in the field, saving time and money for those taking the measurements. The measurements are made within a micro-fluidic channel configured on a substrate of a chip, which is loaded using simple capillary forces. Competitive immunoassay can be performed, with the competing agents being the hormone (e.g., antigen) and hormone-coated latex beads (e.g., both pre-mixed in a methanol solution).

### CROSS-REFERENCE TO PROVISIONAL PATENT APPLICATION

This application claims the benefit of provisional patent application Ser. No. 60/691,699, entitled “Dielectrophoretic Controlled Scat Hormone Immunoassay Technique,” which was filed on Jun. 16, 2005, the disclosure of which is incorporated herein by reference in its entirety.

### TECHNICAL FIELD

Embodiments generally relate to the field of molecular technology, including nanotechnology. Embodiments additionally relate to the field of dielectrophoresis. Embodiments also relate to immunoassay techniques and systems.

### BACKGROUND

Immunoassays are an extremely important tool for a very wide spectrum of applications. It is believed that there is a need for an improved immunoassay for specific antigens, such as corticosterone and progesterone. There is a strong demand for an inexpensive, fast, and portable assay for these particular hormones from behavioral biologists and endocrinologists. Prior art techniques have been implemented, which are non-invasive forms of gathering physiologic stress and reproductive activity data from many different female wild animal species throughout the world. Currently, such immunoassay techniques regularly make use of standard RIA (radioimmunoassay) kits.

Both DNA and hormones can be obtained from animal scat. In fact, scat contains a treasure trove of hormones or hormone metabolites including corticosterone and progesterone. It is the most collectible animal by-product in the wild and can be acquired non-invasively without disturbing the animals. The relatively high concentration of hormones in scat compared to blood allows for effective RIA analysis. The location of scat samples also provides information on individual movements, home range, habitat, and resource use measures. The available information from scat makes collection and analysis a powerful tool for formulating and answering questions in wildlife sciences.

But how does one gather such massive amounts of feces needed to supply the high demands? This isn't an important question in the context of this project, but it is interesting enough to merit mention. After the biologists plan the logistics of an animal study—such as choosing the target species, geographic location, duration, and so forth—a scat detecting team is made up which comprises scat-detection dogs and their handler. The scat-detecting dogs are trained using scenting techniques similar to those used for drugs, bombs, and rescue work. These are high play drive dogs that have a great motivation to find the scat in return for verbal praise, play, and a toss of a tennis ball. They are trained to only find a certain species of scat and ignore all others, and they are used to find scat from whales, bears, owls, and many other wildlife species.

It is believed that a need exists for a chip-based corticosterone and progesterone immunoassay that could be used in the field. The problem with the RIA kits currently in use is that one must do all the processing in a laboratory. Since the kits measure the concentration of the antigen molecules using a radioactive label, a gamma counter is used in the final step to make the measurement. The gamma counter, along with all the other lab equipment needed to prepare the feces sample makes it impossible to carry out these measurements in the field.

In addition, the transportation of the samples is a hindrance, especially when trying to export them from other countries back to the lab. For example, from the time the samples are collected in the wild (e.g., South America) to the time they reach a U.S. laboratory can be up to 1.5 years. The delays, which are mostly bureaucratic, could all be avoided with a portable analysis tool. The cost estimate given for the hormone supplies and analysis for a brown bear study in Alberta were given to be approximately $25 per hormone test. The embodiments disclosed herein would vastly decrease the financial burden on already under funded biological studies.

The immunoassay technique is perhaps the most common clinical method for detecting hormones (and other substances) in a sample. Numerous methods of using immunoassay have been developed, and the fairly recent advances in MEMS have opened up even more options. For any application of immunoassays, one must weigh the pros and cons of the available methods. Some of the various factors to consider are the price (of the initial equipment and each individual test), time and labor requirements, preparation of sample requirements, portability, accuracy, and whether the test is quantitative (giving concentrations) or simply a “yes/no” detection of the presence of the substance. The first four requirements are related. For example, a portable technique requires significantly less time and labor than a technique performed in a lab. Unfortunately, these time/money/labor factors have typically been inversely proportional to the accuracy of the measurements. A few techniques are briefly described, along with their merits and limitations.

Enzyme-linked immunosorbent assay (ELISA) is a method in which an antigen or antibody is connected to an enzyme label. This enzyme typically activates a dye, which can then be detected optically. The accuracy of ELISA ranges from a qualitative (yes/no) measurement (which can be performed simply by observing color changes) to incredibly accurate measurements using optical equipment. In order to get an acceptable accuracy, the ELISA technique must be done in a laboratory setting, where the equipment costs and labor time are both high.

Another technique is radioimmunoassay, which involves an antibody or antigen with a radioactive flag. Radioimmunoassay seems unlikely to be feasible for in the field use, as the equipment, which detects radioactive particles, is difficult to scale down. While it may be possible, it is believed that such a technique would be very expensive and impractical to implement.

Fluorescent immunoassay, as the name implies, involves a flag that fluoresces. It has been miniaturized on a chip but the complexity of detecting the fluorescence make it more feasible in a lab.

Magnetically labeled assays were developed by at least 1977. The first use required superconducting quantum interference devices to sense magnetic labels. Though accurate, these were not portable. A more recent technology, the force amplified biological sensor (FABS) the use of magnetic beads binding to a piezoresistive cantilever. A magnetic field then pulls the beads in some direction, and the change in resistance of the cantilever corresponds to the number of bound beads. This technique is portable and may prove to be quite accurate. However, the relative complexity may keep it unreasonably expensive.

Another approach involves measuring the force required to separate the beads from the chip using optical tweezers. With this method, the bead can be coated with the antigen and then competes with the antigen in the solution for binding antibodies on the chip surface. After the incubation time, optical tweezers are used to force the bead off of the chip, and the force required is related to the concentration of antigens in the solution. This method gave very good results for sensing concentrations as low as 1.45×10ˆ−12 to 1.45×10ˆ−15 mol/L. However, this technique is not portable, nor is the measurement of femtomolar concentrations necessary. The cost of such a system would also likely be too high.

While many ideas for portable, inexpensive chemical detection/measurement techniques have been proposed, few have become commercially produced, and none has become a dominant method. There is still a need for an inexpensive, reliable, accurate, and portable immunoassay technique.

### BRIEF SUMMARY

The following summary is provided to facilitate an understanding of some of the innovative features unique to the embodiments, and is not intended to be a full description. A full appreciation of the various aspects of the embodiments can be gained by taking the entire specification, claims, drawings, and abstract as a whole.

It is, therefore, one aspect of the present invention to provide for an improved immunoassay device and method for testing a biological sample for the presence of particular hormones.

It is another aspect of the present invention to provide for an immunoassay testing device that utilizes dielectrophoresis as a part of the testing process.

The above and other aspects can be achieved as is now described. An immunoassay apparatus on a chip is disclosed, which can quantitatively measure the concentration of hormones (particularly corticosterone and progesterone) in a biological sample. Such an apparatus can be designed to be used in the field, saving time and money for those taking the measurements. The measurements are made within a micro-fluidic channel configured on a substrate of a chip, which is loaded using simple capillary forces. Competitive immunoassay can be performed, with the competing agents being the hormone (e.g., antigen) and hormone-coated latex beads (e.g., both pre-mixed in a methanol solution).

The antibodies are connected to the chip within the micro-fluidic channels between interdigitated capacitors. After some incubation time, as well as the use of positive dielectrophoresis (DEP) to pull the beads to the antibodies, some of the beads will have attached to the antibodies. The number of antigen-antibody bonds connecting the bead and the chip will be inversely related to the number of hormones in the original solution (which block the chip-bead bonds). Using negative DEP, the beads are then increasingly pushed away from the chip substrate. Depending upon the number of bonds, the bonds will break at a certain DEP force (controlled by voltage) and the beads will travel away from the chip. Throughout this procedure, the capacitance is measured. As beads are forced out of the capacitance path, the capacitance will change due to the difference in dielectric constant between the beads and the methanol solution. Although this method makes use of many different physical phenomena, the design and control of the chip is quite simple.

### BRIEF DESCRIPTION OF THE DRAWINGS

FIG. 1 illustrates a chemical diagram depicting the molecular structure of Corticosterone or cortisol metabolite (C₂₇H₄₆O) in accordance with a preferred embodiment;

FIG. 2 illustrates a chemical diagram depicting the molecular structure of Progesterone (C₂₁H₃₀O₂) in accordance with a preferred embodiment;

FIG. 3 illustrates a graph depicting was coupled via long (2.24 nm) spacer arms to bovine serum albumin (BSA) and together with the BSA to form an artificial antigen BBSA;

FIG. 4 illustrates a schematic representation of the MIP principle, which can be adapted for use in accordance with a preferred embodiment;

FIG. 5 illustrates a chemical diagram depicting the molecular structure for a detailed imprint of the molecule theophylline, which can be adapted for use in accordance with a preferred embodiment;

FIG. 6 illustrates a block diagram illustrating a two-compartment model, which can be adapted for use in characterizing one or more embodiments;

FIG. 7 illustrates a graph depicting a graph of kinetic data, in accordance with one or more embodiments;

FIG. 8 illustrates a graph depicting estimated bonding kinetics, which can be implemented in accordance with one or more embodiments;

FIG. 9 illustrates a schematic diagram of a technique for finding the equivalent contact area in accordance with a preferred embodiment;

FIG. 10 illustrates a graph depicting the simulated number of Bonds (10000 total) for varying hormone adherence percentages in accordance with a preferred embodiment;

FIG. 11 illustrates a graph depicting the theoretical number of bonds (10000 total) in accordance with a preferred embodiment;

FIG. 12 illustrates a graph depicting the reduction of variance by increasing coating density, in accordance with a preferred embodiment;

FIG. 13 illustrates a graph depicting the dielectrophoretic force and expected total binding force versus bead radius for a 1, 20 and 100 pN/bond binding force and 05% to 90% coating density in accordance with a preferred embodiment;

FIG. 14 illustrates a graph depicting the “real part” of the Clausius-Mossotti Factor, the frequency-dependant term in Equation (5), depicting the cross-over between positive and negative DEP, in accordance with a preferred embodiment;

FIG. 15 illustrates a graph depicting the cross-over-frequency versus medium conductivity for latex beads, in accordance with a preferred embodiment;

FIG. 16 illustrates a graph of Low-Frequency (+DEP) CM Factor versus Medium Conductivity(S/m), in accordance with a preferred embodiment;

FIG. 17 illustrates a screen shot of an electric field magnitude in accordance with a preferred embodiment;

FIG. 18 illustrates a screen shot of a Magnitude of Gradient of |E|² in accordance with a preferred embodiment;

FIG. 19 illustrates a screen shot of a vertical component of DEP force in association with one or more beveled electrodes in accordance with a preferred embodiment;

FIG. 20 illustrates a schematic diagram of the division of a coplanar plate capacitor into many non-parallel plate capacitors in accordance with a preferred embodiment;

FIG. 21 illustrates a schematic diagram demonstrating that the influence of a neighboring bead on the capacitance is negligible in accordance with a preferred embodiment;

FIG. 22 illustrates a perspective view of a DEPSHIAT chip apparatus, which can be implemented in accordance with a preferred embodiment; and

FIG. 23 illustrates a method for producing the DEPSHIAT chip apparatus depicted in FIG. 22 in accordance with a preferred embodiment.

### DETAILED DESCRIPTION

The particular values and configurations discussed in these non-limiting examples can be varied and are cited merely to illustrate one or more embodiments.

The disclosed embodiments can be implemented in the context of a fast, low-cost immunoassay system on a chip that can quantitatively measure the concentration of hormones (particularly corticosterone and progesterone) in an animal feces sample. It is designed to be used in the field, saving time and money for users taking the measurements. The measurements are made within a micro fluidic channel on the chip, which is loaded using simple capillary forces. Competitive immunoassay is performed, with the competing agents being the hormone (antigen) and hormone-coated latex beads (both pre-mixed in a methanol solution).

The antibodies can be connected to the chip within the micro fluidic channels between interdigitated capacitors. After some incubation time, as well as the use of positive dielectrophoresis (DEP) to pull the beads to the antibodies, some of the beads will have attached to the antibodies. The number of antigen-antibody bonds connecting the bead and the chip will be inversely related to the number of hormones in the original solution (which block the chip-bead bonds). Using negative DEP, the beads are then increasingly pushed away from the chip substrate. Depending upon the number of bonds, the bonds will break at a certain DEP force (controlled by voltage) and the beads will travel away from the chip. Throughout this procedure, the capacitance is measured. As beads are forced out of the capacitance path, the capacitance will change due to the difference in dielectric constant between the beads and the methanol solution. Although this method makes use of many different physical phenomena, the design and control of the chip is quite simple. The chip itself can be referred to as a DEPSHIAT (Dielectrophoretic Scat Hormone ImmunoAssay Technique) chip.

The antigens corticosterone and progesterone are the main target hormones for our DEPSHIAT chip. The concentrations of corticosterone found in feces is an indicator of the animals stress levels approximately 24 hours prior to the deposition of the feces sample. Likewise, the concentration of progesterone is an indicator of reproductive cyclicity and pregnancy in females. These hormones are the two most important signals that the biologists can use in their studies. DNA analysis is also carried out with the same samples, but will not be discussed in this paper.

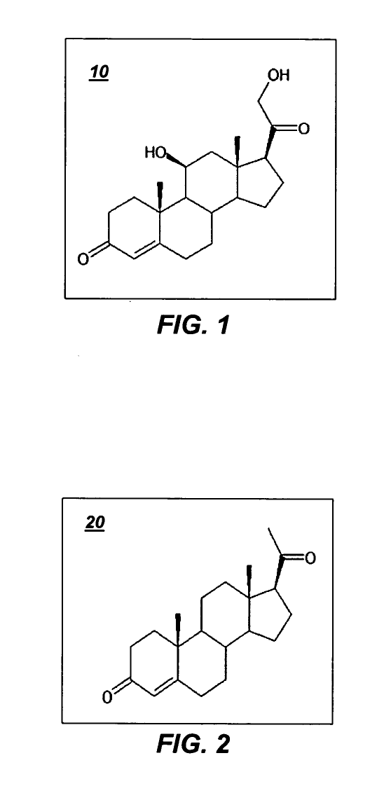

Corticosterone is actually a cortisol metabolite. One could use antibodies in the assay to directly detect cortisol, but it only occurs in very small concentrations in feces. It is a relatively small antigen having a molecular weight of 386.6598 grams/mol. Its molecular formula is C₂₇H₄₆O and its molecular structure is shown in FIG. 1.

FIG. 1 illustrates a chemical diagram depicting a molecular structure 10 of Corticosterone or cortisol metabolite (C₂₇H₄₆O) in accordance with a preferred embodiment. FIG. 2 illustrates a chemical diagram depicting a molecular structure 20 of Progesterone (C₂₁H₃₀O₂) in accordance with a preferred embodiment.

In general, the concentrations that are found in biological samples, such as, for example, scat samples depend on the specific animal, but may range from 5 ng/gram to 5000 ng/gram with an average concentration of 50 ng/gram. A 0.6 gram wet feces sample is mixed with 2 mL methanol and diluted. This gives a range of hormone concentrations of 4.67×10ˆ12 to 1.87×10ˆ15 molecules per 2 mL for corticosterone and 5.74×10ˆ12 to 2.3×10ˆ15 molecules per 2 mL for progesterone. The average expected value may be approximately 4.67×10ˆ13 for corticosterone and 5.74×10ˆ13 molecules per 2 mL for progesterone. This concentration range covers a wide range of species. Even though the DEPSHIAT chip apparatus described herein is designed to deal with these big differences of concentration, a particular DEPSHIAT chip can be custom made for a particular animal for higher accuracy.

The traditional protocol for implementing a competitive assay involves adding the target hormone sample and some labeled hormones of the exact same type to a kit. These hormones compete for binding sites on antibodies that are also present. Normally after a sufficient amount of time, the antigens that have attached are separated from the unattached ones by various ways such as adding charcoal and precipitating out the bound conjugate pairs. Another way is to place the antibodies on the walls of a test tube and dump out the contents after a certain time leaving only the bound antigen behind.

It is also common to add a second larger hormone to the mixture after a certain time, and after it sticks to the bound pair it can be centrifuged. The separated components can then be evaluated by the techniques stated previously. In contrast, our system utilizes prefabricated latex beads coated with covalently bonded antigens. The surface of the chip between the electrodes is coated with antibodies. When the sample and the coated beads are added to the chip together, they compete for the binding sites on the chip surface.

One of the main challenges in the design of the disclosed embodiments involves determining a technique that mimics the separation of the bound and unbound conjugate pairs. A technique must also be determined for performing quantitative measurements of the concentration on-chip without the use of external machines. It is believed that a key to meeting such challenges involves the use of interdigitated fingers that serve dual functions —DEP and capacitive measuring. What is described next is the chemistry of the antigen-antibody binding as well as the coating of the surfaces of the beads and the chip with Ag-Ab.

The DEPSHIAT chip apparatus disclosed herein includes a reactive surface made up of antibodies. These antibodies can be selected to react and bind to their conjugate pair antigen. The bonds between the Ag-Ab are non covalent and may be one or more of the following types: hydrogen bonding, electrostatic forces, Van der Walls, and hydrophobic interactions. To be more precise, the bonds are created between the antigen functional groups and the conjugate amino acids on the antibodies. These bonds are much weaker than the covalent bonds that attach the Ag-Ab to the beads and the chip surface, respectively.

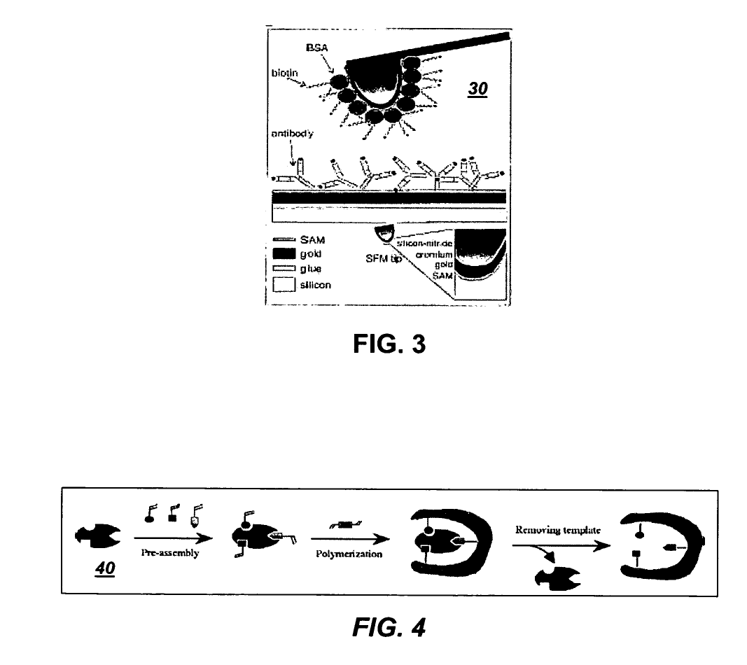

Biotin antigens (actually artificial biotin called biotintylated BSA) can be been covalently attached to the tip of an AFM needle. An experimental setup can be implemented to test the strength of the Ag-Ab bonds between biotin and its conjugate antibody. As an interesting side note, the average bond strength of one Ag-Ab bond can be 60 pN+/−10 pN. In such a scenario, the biotin can be coupled via long (2.24 nm) spacer arms to bovine serum albumin (BSA) and together with the BSA forms an artificial antigen BBSA. FIG. 3, for example, illustrates a graph depicting was coupled via long (2.24 nm) spacer arms to bovine serum albumin (BSA) and together with the BSA to form an artificial antigen BBSA.

Corticosterone and progesterone can be covalently attached to their beads. Latex (polystyrene) beads, however, can be selected because they are readily available from commercial vendors. It is thus possible to obtain antigens coated on the surface of the beads with their functional groups pointing outwards. Certain types of cross linking reagent molecules can also be added to function as spacers, providing more reach and mobility to the antigens. Particular molecules can be attached to micro spheres, either functionalized (e.g. covalently bound to —COOH or —NH2 modified spheres) or protein-coated (e.g. streptavidin- or secondary antibody-coated). In the instance of corticosterone and progesterone, the use of a chemical spacer or carrier molecule (e.g. BSA) may be required to extend the small hormone molecules from the surfaces of the spheres and avoid steric effects. n practice, the coated beads can be adapted for use with the preferred embodiment. As part of the manufacturing step of the DEPSHIAT chip apparatus, it may be necessary to coat the active surfaces of the chip between the interdigitated electrodes with the antibodies. This step can be integrated into the chip manufacturing design.

A method can be implemented for attaching antibodies to a spin coated polystyrene surface via adsorption. The film can be directly spin coated onto a silicon wafer surface. Monoclonal antibodies can be added to the surface and allowed to incubate overnight. It is possible to increase the number of binding sites by controlling the surface roughness. Ultra-flat polystyrene surfaces used for antibody immobilization can increase the number of binding sites and improve the performance of immunoassays.

Whichever antibody immobilization method is finally employed to attach the antibodies to the chip's surface, the issue of dehydrating the surface remains. Most antibodies like to be kept in solution. What is meant by this is that the antibodies retain their shape and hence activity while in solution. When the liquid is removed they tend to shrivel up and collapse and they may not be able to be rejuvenated. It is impossible for our chip to always contain a liquid because then the sample could not be drawn in with capillary action. The solution for this is to add a specially designed chemical to the surface during the fabrication process that acts as a supporter for the antibodies. An immunoassay stabilizer may be desirable, which is a mixture of non-toxic components that greatly enhances the long term stability of many immobilized antigens and antibodies. The solution can be supplied ready to use and, in most cases, can replace current blocking solutions. When properly applied, immunoassay plates or membranes may be completely dried while sacrificing little, if any, of the activity of the fully hydrated protein.

Another method to create an active surface for the antigens to stick to is a newly developed method called molecularly imprinted polymers MIP. These polymers, which can be coated on the surface of a chip, use biomimicry to imitate the binding sites (functional groups) of antibodies. They are said to have a higher chemical and physical stability compared to their biological counterparts. This would allow the surface of our chip to be dried without any problems. They are also shown to withstand higher temperatures and extremes in pH, organic base, and to autoclave treatment.

FIG. 4 illustrates a schematic representation of the MIP principle, which can be adapted for use in accordance with a preferred embodiment. In FIG. 4, the target antigen is depicted with its three monomers shown as indentations. Preassembly involves adding one or more functional monomers that attach to the antigen monomers. The next step is to surround the template with a polymer that contains a cross-linker. The functional monomers will form stronger bonds to the polymer than the rest of the molecule. The antigen is next removed or cleaved from the polymer leaving behind a cavity and an active binding site that is only reactive for the desired antigen. An entire surface covered with these binding sites is preferably able to replace the antibodies used in immunoassay-type binding assays.

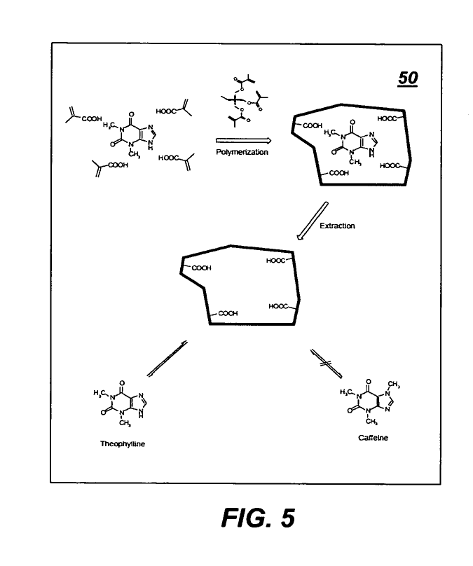

FIG. 5 illustrates a chemical diagram depicting the molecular structure 50 for a detailed imprint of the molecule theophylline, which can be adapted for use in accordance with a preferred embodiment. The molecular structure 50 depicted in FIG. 5 represents a more detailed imprint for the molecule theophylline. The molecular structure 50 of FIG. 5 also indicates that the antigen theophylline can bond with the site, but not the closely related molecule caffeine. A polymer surface can be created for the antigen corticosterone. The anti-corticosterone polymers can be found to be highly specific when compared to a range of structurally similar molecules. These artificial antibodies also displayed cross-reactivities comparable to natural anti-corticosterone antibodies.

A fundamental concern in the design of the disclosed DEPSHIAT apparatus is how the number and distribution of surface antigen-antibody bonds will change with time and concentration. The accuracy of the device can be directly linked to how the fraction of surface coverage correlates to the concentration of antigen in the sample. More antigen in the sample means more of the surface is blocked from binding with the bead and thus less force is measured when attempting to remove the bead from the surface. One can draw on the results of similar antigen-antibody surface reaction studies and models to get an estimate of how ours will react and thus to estimate the feasibility of the disclosed DEPSHIAT device.

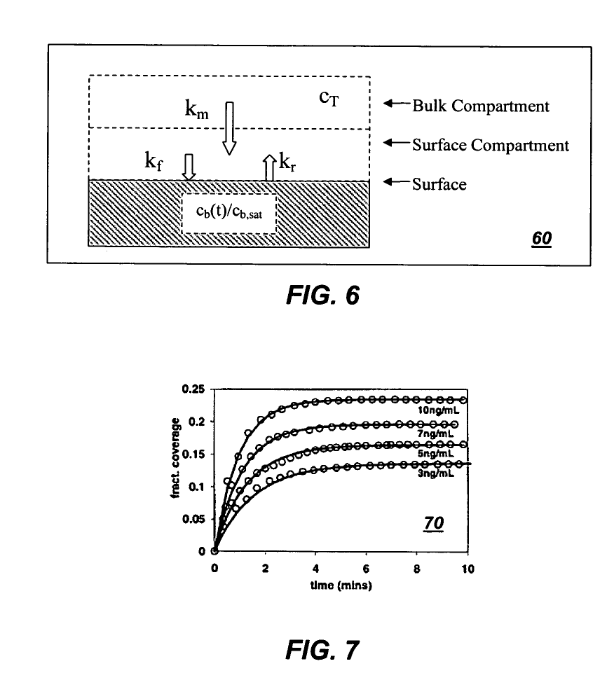

FIG. 6 illustrates a block diagram illustrating a two-compartment model 60, which can be adapted for use in characterizing one or more embodiments. Characterizing the rates at which antigens will attach to a surface that has been coated with antibodies has been studied for years. Binding measurements are usually accomplished in a constantly flowing medium to avoid the impacts of diffusion and convection. The standard model for a flowing medium is referred to as the “well-mixed” model. Unfortunately, our device is not under constant flow so we cannot rely on the well-mixed model. Instead we must rely on a smaller body of work to characterize how the surface bonds will change with time and concentration. One promising candidate for characterizing our system is a two-compartment mode design. A diagram of this model is depicted in FIG. 6 as model 60, and a typical first order differential equation for the model 60 is provided by Equation (1) below:

The two-compartment model 60 depicted in FIG. 6 assumes that a thin compartment encompassing the sensors surface has a lower concentration than the bulk of the solution, which has a constant concentration CT throughout the experiment. Diffusion into the surface compartment is modeled with the rate constant km and the rates at which antigens associate and disassociate are given by rate constants k_{f} and kᵣ respectively.

$$\frac{ⅆ {c}_{b} ( t )}{ⅆ t} = {[ 1 + \frac{{k}_{f} ( {c}_{b , sat} - {c}_{b} )}{{k}_{m}} ]}^{- 1} [ {k}_{f} {c}_{T} ( {c}_{b , sat} - {c}_{b} ) - {k}_{r} {c}_{b} ] \qquad ( 1 )$$

kₘ: Rate constant for diffusion and convection transport
k_{f}: Bimolecular association rate constant
kᵣ: Bimolecular dissociation rate constant
c_{b}: Density of bonds on surface
c_{b,sat}: Density of bonding sites
c_{T}: Concentration in the liquid

FIG. 7 illustrates a graph 70 depicting a graph of kinetic data, in accordance with one or more embodiments. The data depicted in graph 70 of FIG. 7 is based on measurements of the constants for the binding of trinitrobenzene (TNB) to a monoclonal 11B3 anti-TNT antibody on a fiber-optic biosensor. Graph 70 of FIG. 7 and Table 1 below indicate the results of such an experiment. It is interesting to note that after only 4 minutes the fraction of surface coverage seems to approach steady state and the steady state value is unique for each concentration. This distribution would be ideal for our device since it would allow for a large time window in which to measure out data. Another detail to consider is that DEPSHIAT samples may have concentrations in the order of 25 ng/mL which is significantly larger then the samples in the TNB experiment. This could mean that an even greater percentage of the active surface will be covered in our experiment, thus improving its resolution.

TABLE 1
Parameter Estimates from TNB in PBS Experiment

| concn, | k_{f}, | kᵣ | c_{b,sat} | kₘ |
|---|---|---|---|---|
| ng/mL | 10⁵ M⁻¹/s⁻¹ | 10⁻² s⁻¹ | 10⁻⁹ mol/m² | 10⁻⁶ m\hat{ }²/s |
| 3 | 6.8 | 1.3 | 1.5 | 5.6 |
| 5 | 5.2 | 1.3 | 1.7 | 4.7 |
| 7 | 5.0 | 1.5 | 1.0 | 5.5 |
| 10 | 5.0 | 1.7 | 1.8 | 4.8 |

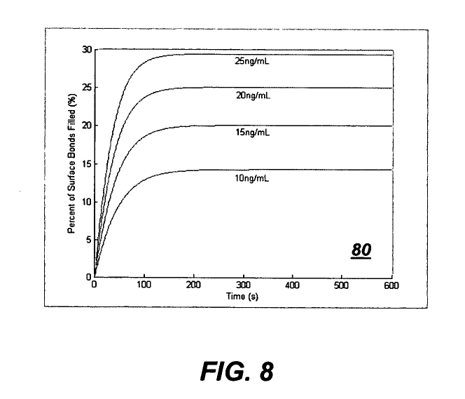

FIG. 8 illustrates a graph 80 depicting estimated bonding kinetics, which can be implemented in accordance with one or more embodiments. Assuming that the rate constants are similar for the DEPSHIAT device, one can numerically solve the two-compartment model equation for the concentrations expected in the DEPSHIAT device. Graph 80 of FIG. 8 depicts the results from these calculations. According to these results, the change in percent coverage with concentration appears well within an estimated measuring capability. It is important to note that this is a rough estimate and the results in practice could differ, but it is a best current estimate of the surface kinetics.

Another factor to consider is that a sample may not be entirely static. Slowly drawing the sample into the device with capillary action and the movement of the beads could have a significant effect on the surface bonding kinetics. Movement of the beads may disturb the surface compartment in the DEPSHIAT model, consequently breaking some of the basic assumptions. If the beads are only allowed near the active surface for a short period of time the effects of their presence can be minimized.

The surface kinetics seems to provide a reasonable chance of success. Estimated bonding kinetics demonstrates that after a relatively short incubation time the surface should reach a quasi-steady state value. This should simplify the measurement process since it will become nearly time independent. The results also showed that we can expect relatively large changes in the percentage of surface bonds with samples of varying concentration. Larger variation with concentration will give the device better resolution. Overall, the surface kinetics lend themselves quite well to the operation of the disclosed DEPSHIAT device.

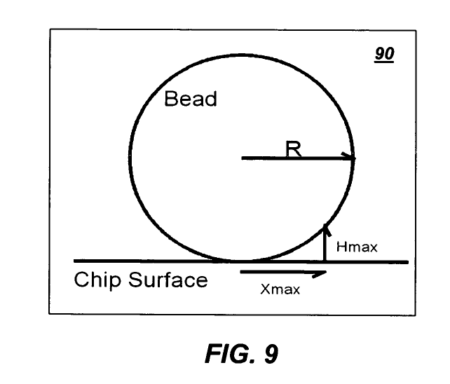

FIG. 9 illustrates a schematic diagram of a technique 90 for determining the equivalent contact area in accordance with a preferred embodiment. The detection of a bound antigen can be found by measuring the force required to remove the antigen-coated bead from the chip surface. The success of the DEPSHIAT method requires a balance of a number of parameters. A synthetic bead coated with antigens is attracted to the surface of the chip via positive DEP. The bead forms a number of non-covalent antigen-antibody bonds with chip surface. Negative DEP can be used to repel the beads from the surface of the chip while the capacitance is monitored, at either at the drive frequency or a lower frequency that has been inductively coupled. The voltage required to remove the bead is proportional to the number of bead/chip bonds, while the change in capacitance is proportional to the number of beads with a particular number of bonds. Ideally, the DEP force is varied over a large range, where the high end corresponds to the force required to remove a bead with the highest expected bond density. One can begin with a first order approximation to the expected bond number.

A first-order model can be composed of a ridged sphere coated with permanently bound and rigid antigens, covering the surface with a random uniform density ƒ_{B}. Likewise, the chip surface can be coated with a random but uniform distribution of antibodies with density ƒ_{c}. ƒ_{B} and ƒ_{c} are generally related to the total number of binding sites by,

$${f}_{B} = \frac{{N}_{A}}{{N}_{total}} \qquad ( 2 )$$

where N_{A} and Nₜₒₜₐₗ are the number of antigens on the bead surface and the total number of possible antigens. In other words, if the surface was mapped to a grid, occupied grid element would represent the presence of an antibody, while

$$ρ = \frac{{N}_{total}}{A}$$

is the packing density representing the grid size.

If A_{B} represents a “bead grid” with packing density ρ_{B}, where ƒ_{B}·A_{B}·ρ_{B} grid elements are occupied with a covalently bound antigen, then the probability of any given grid element forming a bond with the “chip grid” is given by
φ≈ƒ_{B}·ƒ_{C} (3)

With knowledge of an equivalent area of overlap, as well as the statistical chance of forming N bonds, the expected number of bonds can be found. Of course, the bead is not flat, so we must define an equivalent area, A_{equiv}, where A_{equiv}=πR²_{equiv} and R_{equiv} is defined as follows. We can define a distance, hₘₐₓ, which represents the distance from the chip surface to the bead where the probability of forming an antibody-antigen bond goes to zero. To a first order approximation, the probability of bonding is proportional to the chip-bead distance.

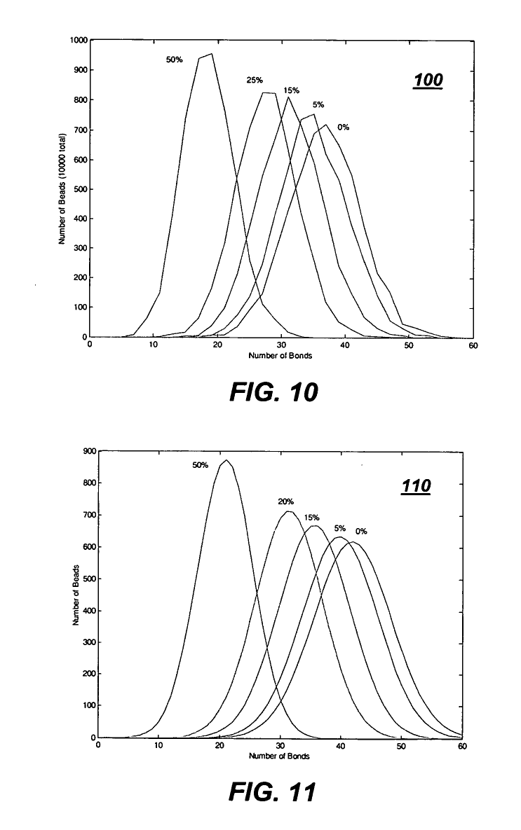

FIG. 10 illustrates a graph 100 depicting the simulated number of Bonds (10000 total) for varying hormone adherence percentages in accordance with a preferred embodiment. FIG. 11 illustrates a graph 110 depicting the theoretical number of bonds (10000 total) in accordance with a preferred embodiment.

At the point of contact, the probability is 1, whereas at a distance hₘₐₓ the probability goes to zero. It can be shown that the height, h, from the chip surface to the bead is given by:

$$h = r ( 1 - \sqrt{1 - \frac{{x}^{2}}{{r}^{2}}} ) \qquad ( 4 )$$

,where r is the bead radius and x the distance from point contact. The probability of bond formation is then

$${P}_{h} = 1 - \frac{h ( x )}{{h}_{max}} \qquad ( 5 )$$

Equation 5 can be used to find an equivalent overlap area by finding an equivalent radius:

$${R}_{equiv} = {\int }_{0}^{{X}_{max}} 1 - \frac{R - \sqrt{{R}^{2} - {x}^{2}}}{{h}_{max}} ⅆ x$$

$${R}_{equiv} = \frac{2}{3} {X}_{max}$$

,where Xₘₐₓ is the distance from bead contact to hₘₐₓ. To find the expected number of bead-chip bonds, we first assume that, out off all potential bonds, each bond has an equal probability of occurrence, φ=ƒ_{B}·ƒ_{C}. Given the probability φ, the probability of forming n bonds out of N trials is given by the binomial distribution:

$$P ( n \vert N ) = \frac{N !}{n ! ( N - n ) !} {{φ}^{n} ( 1 - φ )}^{N - n} \qquad ( 6 )$$

Equation 6 is cumbersome for large values of N, but can be approximated as a normal distribution. The expected number of bonds and variance is given as,
N_{bonds}=ƒ_{B}·ƒ_{C}·ρA_{eqiv}=φ·ρA_{eqiv}σ²=Nφ(1−φ) (7)
so that the approximate normalized probability distribution function is:

$$P ( n ) \frac{1}{\sqrt{2 {πσ}^{2}}} {ⅇ}^{\frac{- {( n - 〈 N 〉 )}^{2}}{2 {σ}^{2}}} \qquad ( 8 )$$

Assuming the presence of the target hormone selectively binds to the chip surface, blocking the antibody sites, the number of bonds can be found by Equation 6, where the probability of bonding has been modified so that the new probability, [symbol] ′, is given by [symbol]
=ƒ_{B}·(1−ƒₕ)·ƒ_{C} (9)
where ƒₕ is the fraction of antibody sites on the chip surface with attached hormone and is proportional to hormone concentration in solution. In reality, the actual probability is more complicated than this, as it is also a function of the orientation of bound antibody, the degree of flexibility of both the bound antibody and antigens, flexibility of the bead surface, etc. To a first order approximation, we can combine all these effects into the coating density parameter, where other factors simply reduce this parameter. If we assume 5000 nm latex beads, with bead and surface antibody/antigen coating density of 10%, Hmax=0.1 nm with a packing density of 3 molecules per square nm, the simulated shift in the probability of bond number can be seen in graph 100 of FIG. 10, while the theoretical curves generated from Equation (8) can be found in graph 110 of FIG. 11.

One can see from the respective graphs 100 and 110 of FIGS. 10 and 11 that the presence of a binding hormone causes a relative linear shift in the bond number probability, as expected. The slight difference between the simulated and theoretical shifts can be explained by the theoretical approximation and sample size. Whereas the true binomial distribution takes on an increasingly skewed form as the distribution maximum approaches zero, the Gaussian approximation does not account for this. To experimentally extract a histogram such as that seen in FIG. 10, the following procedure can be used:
1. Draw environmental sample into chip via capillary action
2. Incubate chip surface (wait)
3. Attract beads to chip surface via positive DEP by application of low-frequency electric field
4. Switch applied electric field to high frequency to induce negative DEP
5. Gradually increase applied voltage while monitoring the change in capacitance.

The change in capacitance is proportional to the change in bead number while the number of bonds is proportional to applied voltage. To extract an accurate measurement it is necessary to apply the same procedure to many beads in parallel while sweeping over a fairly large voltage range. The data of FIG. 10 was generated from the measurement of 10000 random bead/chip pairings. The more beads measured, the more accurate the measurement. As an example, with beads of 5 μm diameter, electrode spacing, width and length of 2 μm, 5 μm and 1000 μm, respectively, with 50 interdigitated electrodes it is possible to measure 10000 beads simultaneously. Of course, large numbers of beads can only be measured in parallel if the concentration of the hormone is large, so that an appreciable fraction of active sites on the chip surface acquire the hormone. The measurement of lower concentration of hormone will lower patterned chip area (and beads), with proportionally less surface area and an increased fraction of bound hormone. To achieve accuracy in the shift of the bond number distribution, the same measurement can be repeated many times. In other words, a 10× decrease in concentration will require a 10× decrease in patterned chip area, and a 10× increase in measurement time. Of course, parameters such as sample size, antibody coating density and particle radius may also be change to decrease the test time for lower concentrations. For example, one can see from Equation (7) that increasing the probability of bond formation will result in more bonds, which will decrease the variance.

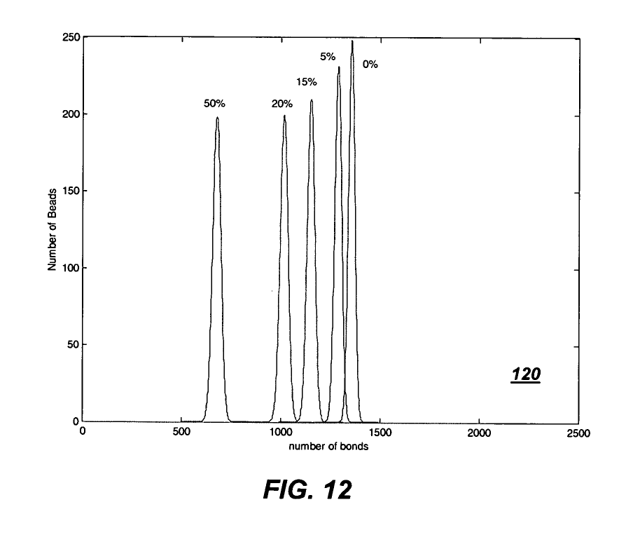

Decreasing the variance will allow fewer measurements to be taken. As an example, consider FIGS. 10 and 11. The packing density was assumed at 3 molecules per nanometer. This is actually a fairly high packing density and is generally about 1 order of magnitude smaller. On the other hand, the coating densities of 10% for bead and 10% for chip are lower than normal, where monoclonal, near-100% coating is feasible. Ideally, what is wanted is measurement with low variance. One can see from equation 7 that the variance is related to the total number of possible bond sites as well as the coating density. For a given packing density, the variance is minimized for antibody and antigen coating densities approaching 1. This can be seen in graph 120 of FIG. 12, where the coating density is now 90% for both bead and chip, and the packing density brought down to a more realistic 0.3 molecules per square nanometer. FIG. 12 generally illustrates a graph 120 depicting the reduction of variance by increasing coating density, in accordance with a preferred embodiment;

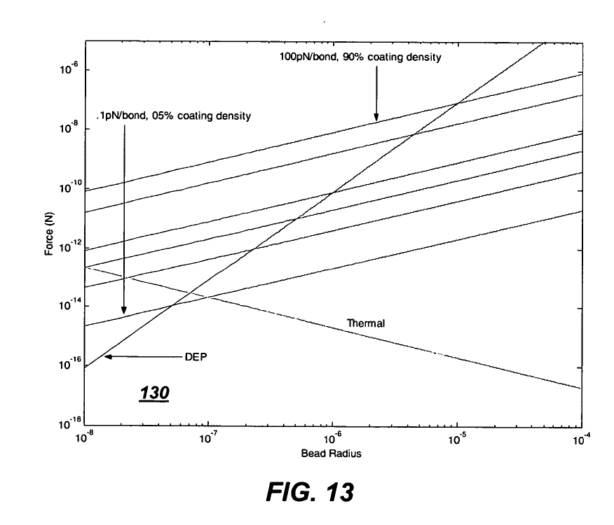

FIG. 13 illustrates a graph 130 depicting the dielectrophoretic force and expected total binding force versus bead radius for a 1, 20 and 100 pN/bond binding force and 05% to 90% coating density in accordance with a preferred embodiment. The success of the DEPSHIAT method requires the ability to pull the bound bead off the surface of the chip for a variety of different target molecules. Fortunately, the DEP force scale as R³, whereas the number of bonds scales as R², so as the bead size is increased the DEP force will eventually overtake the bond strength and the bead will be removed. We turn now to a more detailed look at the DEP force.

When a particle is suspended in a solution and subjected to an electric field, the electric field induces a polarization in the particle. If the field is homogeneous, the induced dipole aligns in the direction of the field. If the field is inhomogeneous, the particle will feel a force. The direction of the force is determined by the dielectric properties of the particle and suspension. If the particle is more polarizable than the surrounding medium, the particle will feel a force in the direction of increasing field gradient, which is termed Positive DEP. On the other hand, negative DEP results when the medium is more polarizable than the particle. At low frequencies, charge accumulation at the particle/medium boundary contributes to the induced dipole, which is referred to as the Maxwell-Wagner interfacial polarization and is of course a function of the particle and medium conductivity. As the frequency is increased, this term of the polarization has increasingly less of an effect, as the mobile charges do not have time to move an appreciable distance. For the case of a spherical particle, the time-averaged DEP force is given by:

$${F}_{dep} = 2 π {r}^{3} {ɛ}_{0} {ɛ}_{m} Re [ \frac{{ɛ}_{p}^{*} - {ɛ}_{m}^{*}}{{ɛ}_{p}^{*} + 2 {ɛ}_{m}^{*}} ] ∇ {E}^{2} \qquad ( 10 )$$

For any geometry other than a sphere or ellipsoid calculating the DEP force is not trivial, and the applicability of Equation (10) requires the particle radius to be small compared to the changes in the gradient of the energy density (∇E²). This is certainly not the case for the proposed electrode and bead geometry, as the bead will be larger than the inter-electrode spacing. However, for the case of coplanar electrodes, finite element simulation has found that the maximum DEP force occurs when the particle radius is on the same order as the electrode width. A general conclusion is that the force calculated from equation 10 will give an under-estimate of the force (about 20%), as the equation does not include higher-order moments which become increasingly important for large bead sizes. FIG. 13 generally illustrates an expected DEP force given by Equation (10) versus the expected bond strength given by F_{bond}=NF_{A}, where F_{A} is the antibody-antigen bond strength and N is the expected number of bonds given by Equation 2. Packing density was assumed at 0.3 molecules per square nanometer and antibody/antigen covering density ranging from 5% to 90%. The y-component of the gradient of energy density was assumed at 10¹⁷V²/m³. This value is based on simulated values compared to experimental data and verified. A finite element analysis can also be performed via custom MATIab programs and obtained values that agree to within the same order of magnitude.

As one can see from FIG. 13, bead sizes from 100 nm to about 10 μm cover a wide range of force required to remove the bead from the chip surface. Ideally, monoclonal (i.e. 100%) coating density on 10 μm bead should provide a high level of accuracy. Random thermal motion becomes increasingly important for smaller bead sizes, and is given roughly as

$${F}_{thermal} \approx \frac{{K}_{B} T}{2 r} \qquad ( 11 )$$

Thermal motion is not expected to be a problem, as larger bead sizes will be used. The thermal force can be seen in comparison to the DEP and binding forces depicted in graph 130 of FIG. 13. Although graph 130 of FIG. 13 was generated with specific antigen and antibody coating densities, the general conclusion is that the DEP force is always capable of removing the bead for realistic field gradients generated by micro fabricated electrodes, as it overtakes the bond force as the particle size is increased.

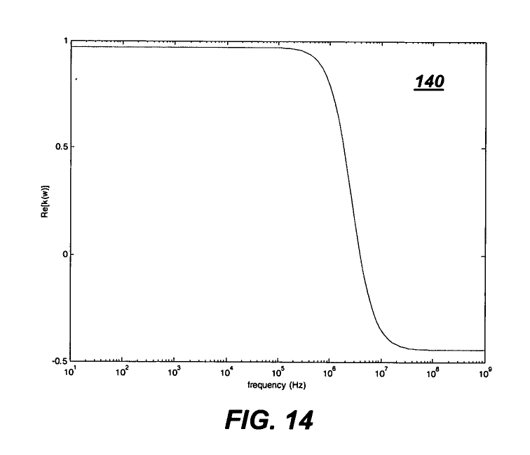

FIG. 14 illustrates a graph 140 depicting the “real part” of the Clausius-Mossotti Factor, the frequency-dependant term in Equation (5), depicting the cross-over between positive and negative DEP, in accordance with a preferred embodiment. It is evident from Equation (10) that the DEP force is dependant on real part of the Clausius-Mossotti (CM) factor

$$\frac{{ɛ}_{p}^{*} - {ɛ}_{m}^{*}}{{ɛ}_{p}^{*} + 2 {ɛ}_{m}^{*}} \qquad ( 12 )$$

The value of the CM factor determines the sign of the force. For positive values, the force is directed along the direction of maximum field gradient. For the DEPSHIAT application, we require positive DEP to initially attract the beads to the surface and negative DEP to perform the force measurement. The CM factor is determined by the complex permittivity, which can be expressed as,

$${ɛ}^{*} = ɛ - \frac{σ}{ω} ⅈ \qquad ( 13 )$$

where σ is the conductivity of the material. Equation (12) warrants special attention, particularly for our desired application. The relative permittivity and conductivity of the bead and the medium determines a cross over frequency, where the DEP force transitions from positive DEP to negative DEP. This can be seen in FIG. 14 for Latex beads in Methanol.

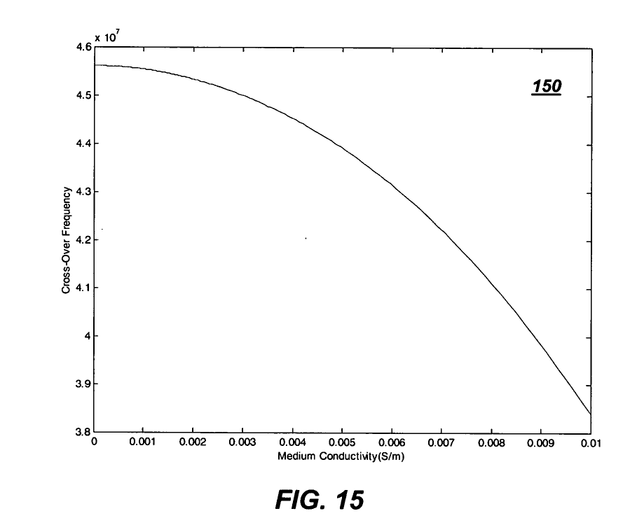

FIG. 15 illustrates a graph 150 depicting the cross-over-frequency versus medium conductivity for latex beads, in accordance with a preferred embodiment. The transition from positive DEP to negative DEP is critically dependant on the conductivity of the bead and medium, indeed the entire effect is possible because of the conductivity. Our application relies on the ability to use both positive and negative DEP. Because environmental samples will be variable in conductivity, we must be sure to design the system such that both positive and negative DEP is assured. The real part of the CM factor is given by:

$$Re [ CM ] = [ \frac{( {ɛ}_{p} - {ɛ}_{m} ) ( {ɛ}_{p} + 2 {ɛ}_{m} ) - \frac{1}{{ω}^{2}} ( {σ}_{m} - {σ}_{p} ) ( {σ}_{m} + {σ}_{p} )}{{( {ɛ}_{p} + 2 {ɛ}_{m} )}^{2} + \frac{1}{{ω}^{2}} {( {σ}_{m} + {σ}_{p} )}^{2}} ] \qquad ( 14 )$$

One can see that as the frequency is increased, the conductivity becomes increasingly insignificant. As long the condition εₚ<εₘ is satisfied, negative DEP will be insured for high frequencies. The cross-over frequency can be found from equation 14 and is given by:

$$ω = \sqrt{\frac{( {σ}_{m} - {σ}_{p} ) ( {σ}_{m} + {σ}_{p} )}{( {ɛ}_{p} - {ɛ}_{m} ) ( {ɛ}_{p} + 2 {ɛ}_{m} )}} \qquad ( 15 )$$

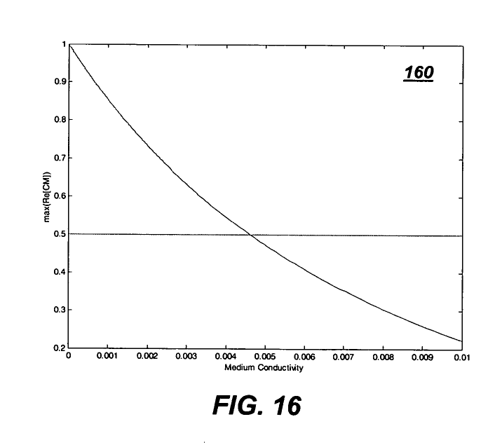

FIG. 15 depicts a graph 150 indicating the cross over frequency plotted against the medium conductivity. One can see from graph 150 of FIG. 15 that the cross over frequency remains between 3.8 and 2.6 MHz for conductivities below the conductivity of latex, estimated from DEP research as being 0.0185 S/m. This means that a frequency greater than 5 MHz will always result in negative DEP. More important, however, is the strength of positive DEP for low frequencies. One can see from Equation (9) that for medium permittivity greater than particle permittivity, (which is the case for Latex beads in Methanol), if the medium conductance is greater than the particle conductance, the resulting cross-over frequency is imaginary. This simply means the cross-over frequency does not exist, and the DEP force is always negative. FIG. 16 plots the maximum low-frequency positive DEP force versus medium concentration. FIG. 16 illustrates a graph 160 of Low-Frequency (+DEP) CM Factor versus Medium Conductivity(S/m), in accordance with a preferred embodiment.

One can see from graph 160 of FIG. 16 that for medium conductivities greater than 0.00045 S/m, the positive DEP force will be greater or equal the negative DEP force for saturated frequencies. The significance of this is as follows. We wish to attract a bead to a specific location of the chip, and propose to use positive DEP. For an interdigitated, coplanar electrode arrangement, the DEP force generated is due to the high field gradient at the electrode corners. For particles smaller than the electrode gap distance, the particles will be attracted opposing corners. Ideally, we would like all particles to line up between the electrodes. For this to occur, the bead must be larger than the electrode gap distance, and there must exist a strong positive DEP force. Yet we also require a strong negative DEP force to remove the bead later. As long as the medium conductivity is less than the measured conductivity of the latex beads (0.0045 S/m), this is assured. If the environmental samples cannot be brought to within this range, more conductive beads must be used.

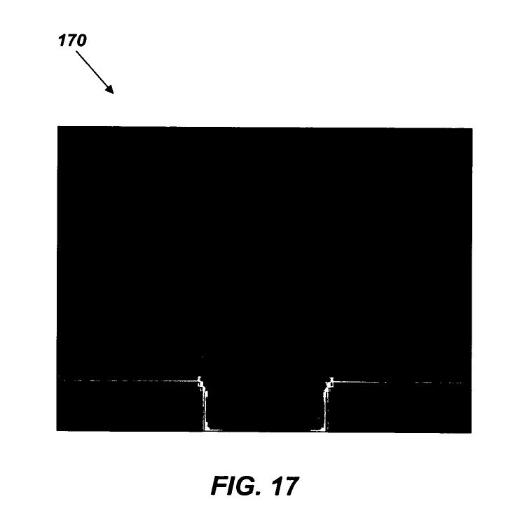

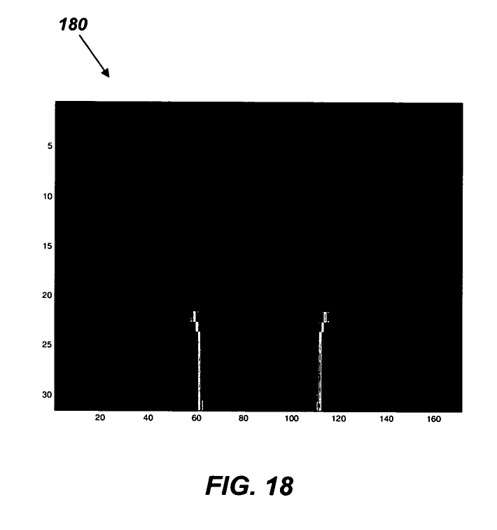

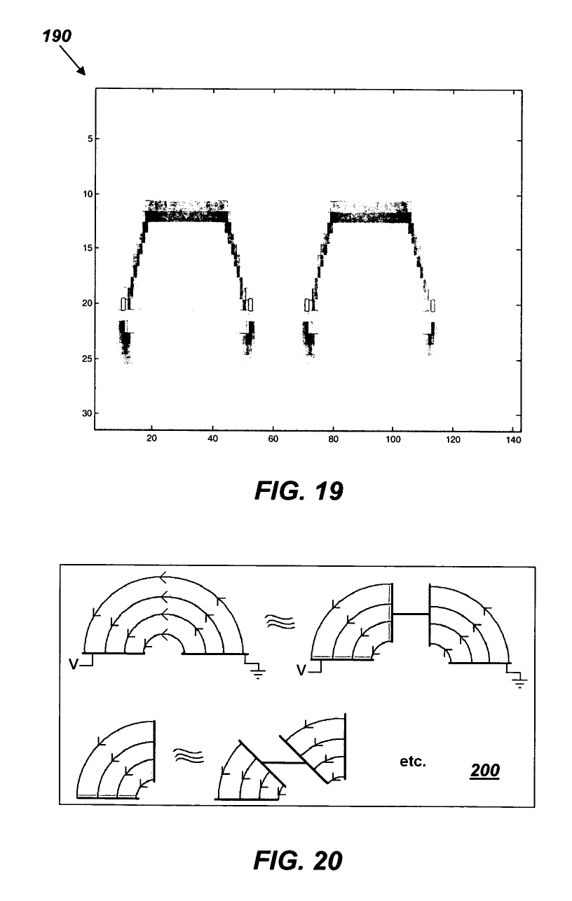

FIG. 17 illustrates a screen shot 170 of an electric field magnitude in accordance with a preferred embodiment. FIG. 18 illustrates a screen shot 180 of a Magnitude of Gradient of |E|² in accordance with a preferred embodiment. FIGS. 17 and 18 show the relative magnitude of the electric field and field gradient, respectively, for a cross section perpendicular to electrode orientation. FIGS. 17 and 18 indicate that the DEP force radiates from electrode corners, as expected. This actually represents a system between electrodes designed to maximize capacitance and the ability to attract the bead to a specific location. Ideally, one would like to maximize the capacitance change as a bead leaves the electrode gap. This could most easily be accomplished by forming a space between the electrodes that the bead would fill. Of course, the bead will be pulled to areas of high field gradient and would therefore not make it into the bottom of the gap. This can be seen in FIG. 19, which illustrates a screen shot 190 of a vertical component of DEP force in association with one or more beveled electrodes in accordance with a preferred embodiment.

As the bevel is made shallower, so that the strong DEP force emanating from the bottom electrode corners draws the bead into the gap, the resulting change in capacitance decreases. One very strong priority for the DEPSHIAT chip is ease of fabrication and thus low unit cost. Beveling the electrodes to increase the change in capacitance has thus been dismissed for simplicity of fabrication. Because the bead size is on the order of the electrode size, and the permittivity change between latex and methanol is high (2.5 vs. 33), an appreciable capacitance change is expected.

Coplanar capacitors exhibit a substantially lower capacitance than parallel plate capacitors. The calculation for the capacitance is also much less straightforward, but has been calculated as follows:

$$C = \frac{L {ɛ}_{r} {ɛ}_{o} ln ( \frac{8 w}{d} )}{π}$$

Where L is the length of the electrode, w is the width, and d is the gap. In order to arrive at this equation, conformal (elliptical) mapping was done to map the coplanar capacitor to an equivalent parallel plate capacitor.

A problem with this solution is that it is difficult to factor in the effect a bead would have between the two plates, since the bead would also have to be mapped. Instead, we make an approximation assuming the field lines are circular, as opposed to elliptical. Based on the property of (parallel plate) capacitors that a capacitor with gap d is equivalent to two capacitors in series whose gaps add up to d, we then treat our coplanar capacitor as many non-parallel plate capacitors in series, as depicted in FIG. 20.

FIG. 20 illustrates a schematic diagram 200 of the division of a coplanar plate capacitor into many non-parallel plate capacitors in accordance with a preferred embodiment. As mentioned, this is an approximation, as the field lines from the coplanar electrodes would be circular only for very small angles. The capacitance of non-parallel plates can be calculated to be as follows:

$$C = \frac{L {ɛ}_{r} {ɛ}_{o} ln ( \frac{d}{d - 2 h tan ( \frac{B}{2} )} )}{B}$$

Where B is the angle between the plates, h=(r₂−r₁)cos(B/2), and d=r₂sin(B/2) r₂ and r₁ are the distance from the center of curvature to the outside of the electrode and to the inside of the electrode, respectively. Note that if one uses this equation to calculate the capacitance between coplanar plates (B=Π), one will get the same answer as if calculating the series capacitance of n planes with angle Π/n. The reason we split up the capacitor is to aid in calculating the effects of a bead within the capacitor. When using electrode dimensions of width 5 μm and gap 2 μm, this approximation gives

$$\frac{C}{{ɛ}_{r} {ɛ}_{o} L} = 1.14 .$$

The mapped equation gives a value of 0.954, or an error of about 20%. Note that for both of these calculations, the material above and below the capacitor can be as the same, and the capacitance calculated is actually double what is depicted in FIG. 20).

The next step is to calculate the capacitance assuming a bead is now between the electrodes. With parallel plate capacitors this is quite easy, since no matter where a volume with a different dielectric constant is (assuming it is within the plates), it will affect the capacitance the same. With non-parallel plates, however, a volume closer to the smaller gap will more greatly affect the capacitance. This is because the field lines are stronger where the plates are closer. For the example given above, about half of the capacitance is due to the first 1.5 microns of our plate, and the other 3.5 microns accounts for the other half. To account for this, we can further divide the non-parallel plate capacitor in many capacitors in parallel. Each cylindrical sliver (of the field) will then have a certain percent of its volume within the bead. In cylindrical coordinates, the surface area of the cylinder that is within the latex bead (whose bottom side is at the center of the cylinder) is given by the integral:

$$4 {\int }_{{θ}_{min}}^{π / 2} {\int }_{0}^{\sqrt{( 2 rs ) rc sin ( θ ) - {rc}^{2}}} rc ⅆ z ⅆ ω = 4 {\int }_{arcsin ( rc / ( 2 rs ) )}^{π / 2} rc \sqrt{( 2 rs ) rc sin ( θ ) - {rc}^{2}} ⅆ ω$$

where rc is the radius of the cylinder and rs is the radius of the sphere. Next, the volume within the bead of each cylinder at a distance of rc from the center of the capacitor and thickness Δr is found simply by multiplying the surface area above by Δr. If we take the length of each capacitor to be equal to the radius of the bead (so the bead “fills” the capacitor), the capacitance of each Δr sliver, assuming we make Δr small enough that the capacitance from rc to rc+Δr is the same, is then given by:

$$C ( rc ) = C ( below ) + C ( above ) ;$$

$$C ( above ) = \frac{1}{\frac{1}{C ( bead )} + \frac{1}{C ( no\_bead )}}$$

$$C ( below ) = \frac{1}{2} \frac{L {ɛ}_{SiO 2} {ɛ}_{o} ln ( \frac{d}{d - 2 h tan ( \frac{B}{2} )} )}{B}$$

$$C ( bead ) = \frac{L {ɛ}_{bead} {ɛ}_{o} ln ( \frac{d}{d - 2 h tan ( \frac{B}{2} )} )}{{B}_{b}}$$

$$C ( no\_bead ) = \frac{L {ɛ}_{meth} {ɛ}_{o} ln ( \frac{d}{d - 2 h tan ( \frac{B}{2} )} )}{{B}_{m}}$$

$${B}_{b} = \frac{Vbead}{Vcylinder} B$$

$${B}_{m} = ( 1 - \frac{Vbead}{Vcylinder} ) B$$

Where C(below) is the capacitance through the chip (assuming SiO2 is thick), C(bead) is the capacitance through the bead, and C(no bead) is the capacitance through the methanol solution for each Δr section. The ratios involving Vbead and Vcylinder are the amount of each Δr sliver that are filled or not filled by the methanol solution. B goes from 0 to Π (and is therefore only in the top half plane). The capacitance of each section is therefore controlled by the volume ratio and by d and h, which vary based on rc. When we sum up all of the Δr sections, we get the total capacitance.

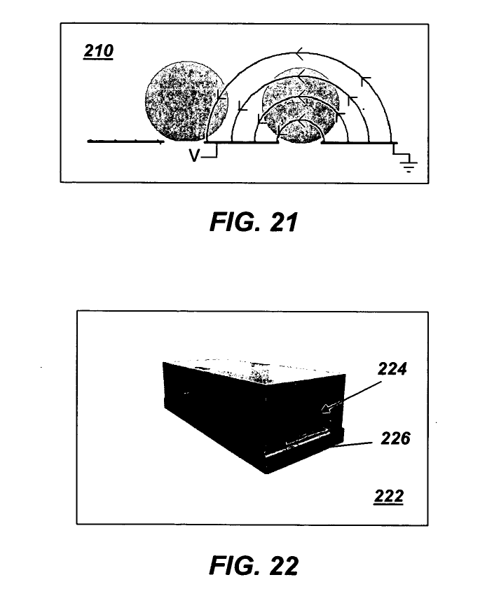

One can then compare these to the values without a bead. Using the dimensions mentioned above (gap=2 micron, width=5 micron, rs (bead radius)=5 micron) and ε_{SiO}_{2=3.9}, ε_{bead}=255, and εₘₑₜₕₐₙₒₗ=33.1 [22], we calculated capacitances with and without the bead as 0.322 fF and 0.931 fF, respectively, for each section of a capacitor with length=5 μm. When extended to a length of 1000 μm and multiplied by 50 interdigitated electrodes, we get values of 3.22 pF and 9.31 pF. These calculations were made assuming that the capacitance between two electrodes was affected only by the bead that it is bonded between the electrodes. As can be seen in FIG. 21, this is likely a fine assumption, because neighboring beads only slightly intersect the field. FIG. 21 illustrates a schematic diagram 210 demonstrating that the influence of a neighboring bead on the capacitance is negligible in accordance with a preferred embodiment. The method to detect this change in capacitance is yet to be determined, although techniques are available. The change of approximately 3× is large enough to detect. If the absolute capacitance is found to be too low to detect, however, the shape of the electrodes could be manipulated to provide more capacitance. One idea previously mentioned is to have the electrodes either triangular or trapezoidal in shape. This would increase the capacitance, however the fabrication would be more complex and costly.

FIG. 22 illustrates a perspective view of a DEPSHIAT chip apparatus 222, which can be implemented in accordance with a preferred embodiment. The device 222 can be implemented as an open ended micro fluidic channel 224 with electrical pads to interface external electronics. The apparatus 222 is generally formed on a substrate 226. The channel 224 can be inert beside a thin monolayer strip of antibodies that can selectively react with the sample and beads. To avoid the complicated chemistry of attaching antigens to the surface of the beads, functionalized beads can be provided by a third party supplier.

The fabrication process for this device can be broken down into three main sections. These sections can create isolated interdigitated electrodes, attaching a layer of antibodies in the electrode trench, and making the PDMS micro fluidic channel 224.

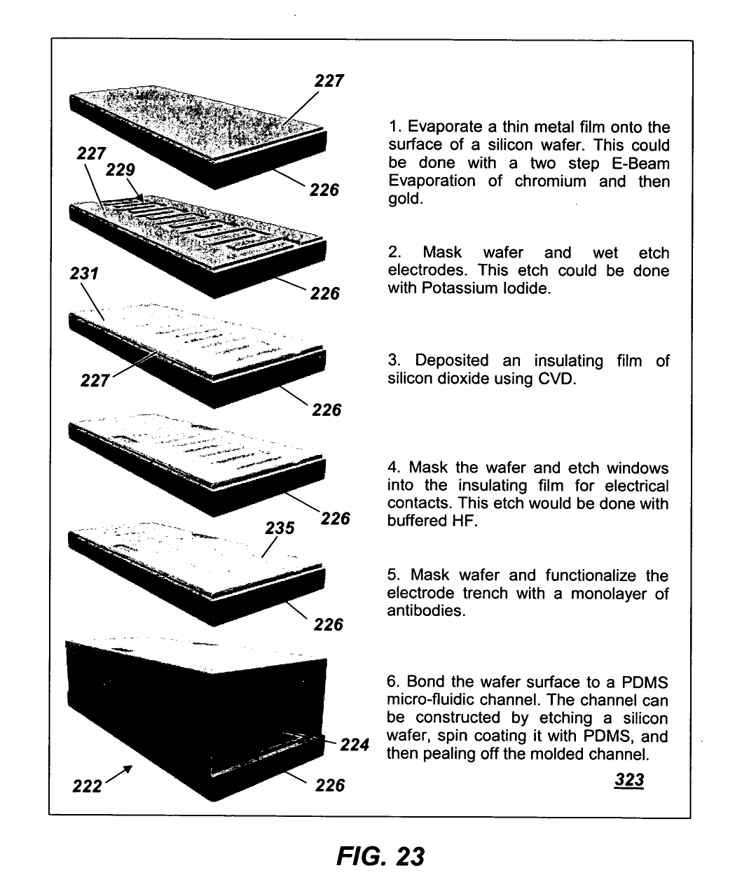

FIG. 23 illustrates a method 323 for producing the DEPSHIAT chip 222 apparatus depicted in FIG. 22 in accordance with a preferred embodiment. Note that in FIGS. 22-23, identical parts or elements are generally indicated by identical reference numerals. Additionally, each process step depicted in FIG. 23 is respectively associated with an adjacent graphical representation of particular elements related to that that particular process step. In general, according to the method depicted in FIG. 23, electrodes can be formed by depositing a metal film on a silicon substrate and etching the desired electrode pattern. The deposition could be accomplished utilizing a two step e-beam evaporation. First, a thin chromium adhesion layer would be put down on the substrate and then the gold would be evaporated on to a thickness of 0.2 um. After cleaning the wafer photoresist would be spin coated on and soft baked. The patterning could then be accomplished with an LPG mask and then developed. The high tolerances of the device means that an isotropic wet etch would be suitable. This etch could be done with Potassium Iodide. The remaining photo resist would then be wet stripped and a thin insolating film of silicon dioxide could then be deposited with a PECVD. To access the underlying electrodes windows in the oxide would then be lithographically patterned and etched with buffered HF. A final wet strip would clean the wafer and prepare it for chemical fictionalization.

The antibodies need to be placed between the electrodes. A mask will define these active areas. The fabrication steps for this were described earlier as well as the MIP option for a synthetic antibody layer. The micro fluidic channel would be created using PDMS micro channel molding. First a silicon wafer would be etched with the negative of the channel and then the wafer would be spin coated with PDMS. After cross-linking the PDMS it is then pealed off and adhesively attached over the functionalized electrodes. Once this is completed the device is ready to be tested and calibrated.

The fabrication process or method 323 thus can be summarized as follows. First, as indicated at Step 1 in FIG. 23, a thin metal film 227 can be evaporated onto the surface of a silicon wafer substrate 226. This can be accomplished with a two-step E-beam evaporation of chromium and then gold. Thereafter, as indicated at Step 2, the silicon wafer substrate 226 can be masked and the electrodes 229 wet etched. Next, as depicted at Step 3, an insulating film 231 of silicon dioxide can be deposited using a CVD process. Thereafter, as illustrated at Step 4 the wafer can be masked and windows etched into the insulating film for electrical contacts. Next, as depicted at Step 5, the wafer can again be masked and the electrode trench functionalized with a monolayer of antibodies 235. Finally, as indicated at Step 6, the wafer surface can be bonded to the PDMS micro-fluidic channel 224 depicted in FIG. 22. The channel 224 can be constructed by etching a silicon wafer, spin coating it with PDMS, and then pealing off the molded channel 224.

Based on the foregoing, it can be appreciated that a fast, low-cost immunoassay system-on-a-chip has been disclosed, which can quantitatively measure the concentration of hormones. The implementation is fundamentally simple, yet synthesizes a number of separate techniques. First, we have found that is possible and relatively straight forward to coat antibodies and antigens on the chip and bead surface. Second, we determined the binding kinetics of the antigen/antibody bonds, as well as the expected bond number for varying concentrations of target antigens and bead radius. Third, we evaluated the dielectrophoretic force and its suitability as a mechanism for controlled force measurement, with encouraging results. Fourth, the expected change in capacitance was found and determined to be well within a measurable range. Finally, the layout and fabrication for a chip is detailed.

Although we have found examples of binding antibodies to a bead surface, there are no prior techniques for our specific target hormones. We anticipate that this is not a substantial problem, as many similar target molecules have been verifiably coated. The rate constants for binding kinetics must be experimentally determined, and again, we have no specific examples for our target hormones. Our simple model for determining the number of bonds does not take into account a variety of potential effects, such as antibody flexibility and orientation, although it was shown that the technique is viable for a wide range of coating densities and bond strengths. For low concentrations of target antigens, there may not be a significant number of blocked sites based on the two-compartment model (no flow).

A potential method for increasing the movement of the solution involves using the beads to mix the solution. We would cycle between attracting the beads to create bonds and the pushing the beads away. Every time we forced them away, we would measure the force required to do so. It is expected that as we go through cycles, more free antigens would bind, so the force would drop. The DEP force was not calculated exactly for the specific geometry yet was shown to be within the desirable operating range. Hydrodynamic and thermal heating were not specifically taken into account. The calculated change in capacitance was also based on a simplified model, so the exact value of the capacitance may not match the final chip, although the change in capacitance should remain approximately the same.

There is much room for future work for our chip design, besides addressing the potential problems explained above. One concern is packaging, although its possible the chip die could itself function as the package. Another possibility, which would decrease the number of beads needed, and therefore reduce the cost, is integrating the antigen-coated beads into the chip channel. However, this may be at the expense of complicating the fabrication process. Perhaps the most important aspect of the chip is its ability to be mass produced cheaply. The integration of the chip into main-stream use will require a robust and fault-tolerant design with the ability to perform a read-out with portable on-site and battery operated equipment.

It will be appreciated that variations of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

What is claimed is:

1. An immunoassay testing apparatus, comprising:
a chip comprising a substrate upon which a plurality of interdigitated capacitors are formed; and
at least one micro-fluidic channel configured from said substrate, wherein antibodies are connected to said chip within said at least one micro-fluidic channel between said plurality of interdigitated capacitors, such that a biological sample can be loaded into said at least one micro-fluidic channel using a capillary force in order to perform an immunoassay upon said biological sample and thereby quantitatively measure the concentration of hormones in said biological sample.

2. The apparatus of claim 1 further comprising a mechanism for performing said immunoassay, wherein said immunoassay is based on a plurality of competing agents, which comprise said hormones and a plurality of hormone-coated latex beads.

3. The apparatus of claim 2 wherein said hormones comprise antigens.

4. The apparatus of claim 2 wherein said plurality of hormone-coated latex beads are pre-mixed in a methanol solution.

5. The apparatus of claim 4 wherein after a particular incubation time, a positive DEP is used to pull said plurality of hormone-coated latex beads to said antibodies so that a particular quantity of beads will attach to said antibodies.

6. The apparatus of claim 5 wherein a number of resulting antigen-antibody bonds connecting said plurality of hormone-coated latex beads and the chip is inversely related to a number of hormones in an original solution associated with said biological sample.

7. The apparatus of claim 6 wherein a negative DEP is utilized to increasingly push said plurality of hormone-coated latex beads away from said substrate.

8. An immunoassay testing apparatus, comprising:
a chip comprising a substrate upon which a plurality of interdigitated capacitors are formed; and
at least one micro-fluidic channel configured from said substrate, wherein antibodies are connected to said chip within said at least one micro-fluidic channel between said plurality of interdigitated capacitors, such that a biological sample can be loaded into said at least one micro-fluidic channel using a capillary force in order to perform an immunoassay upon said biological sample and thereby quantitatively measure the concentration of hormones in said biological sample; and
a mechanism for performing said immunoassay, wherein said immunoassay is based on a plurality of competing agents, which comprise said hormones and a plurality of hormone-coated latex beads.

9. The apparatus of claim 8 wherein said hormones comprise antigens.

10. The apparatus of claim 8 wherein said plurality of hormone-coated latex beads are pre-mixed in a methanol solution.

11. The apparatus of claim 10 wherein after a particular incubation time, a positive DEP is used to pull said plurality of hormone-coated latex beads to said antibodies so that a particular quantity of beads will attach to said antibodies.

12. The apparatus of claim 5 wherein a number of resulting antigen-antibody bonds connecting said plurality of hormone-coated latex beads and the chip is inversely related to a number of hormones in an original solution associated with said biological sample.

13. The apparatus of claim 12 wherein a negative DEP is utilized to increasingly push said plurality of hormone-coated latex beads away from said substrate.

14. An immunoassay method, comprising:
providing a substrate;
configuring a chip from said substrate;
forming a plurality of interdigitated capacitors upon said substrate;
configuring at least one micro-fluidic channel from said substrate;
connecting antibodies to said chip within said at least one micro-fluidic channel between said plurality of interdigitated capacitors; and
loading a biological sample into said at least one micro-fluidic channel using a capillary force in order to perform an immunoassay upon said biological sample and thereby quantitatively measure the concentration of hormones in said biological sample.

15. The method of claim 15 f performing said immunoassay based on a plurality of competing agents, which comprise said hormones and a plurality of hormone-coated latex beads.

16. The method of claim 15 wherein said hormones comprise antigens.

17. The method of claim 15 wherein said plurality of hormone-coated latex beads are pre-mixed in a methanol solution.

18. The method of claim 17 further comprising utilizing a positive DEP after a particular incubation time to pull said plurality of hormone-coated latex beads to said antibodies so that a particular quantity of beads attaches to said antibodies.

19. The method of claim 18 wherein a number of resulting antigen-antibody bonds connecting said plurality of hormone-coated latex beads and the chip is inversely related to a number of hormones in an original solution associated with said biological sample.

20. The method of claim 19 further comprising utilizing a negative DEP to increasingly push said plurality of hormone-coated latex beads away from said substrate.
